(19) United States
(12) Patent Application Publication
(10) Pub. No.: US 2020/0022819 A1
(43) Pub. Date: Jan. 23, 2020

(54) Multi-Matrix Composite Prosthetic Socket and Methods of Fabrication

(71) Applicant: LIM Innovations, Inc., San Francisco, CA (US)

(72) Inventors: Garrett Ray Hurley, San Francisco, CA (US); Loren Maxwell Brock, Sebastopol, CA (US)

(73) Assignee: LIM Innovations, Inc., San Francisco, CA (US)

(21) Appl. No.: 16/516,143

(22) Filed: Jul. 18, 2019

Related U.S. Application Data
(60) Provisional application No. 62/736,876, filed on Sep. 26, 2018.
(60) Provisional application No. 62/701,310, filed on Jul. 20, 2018.

(51) Int. Cl. A61F 2/50; A61F 2/80; B29C 70/08; B29C 70/44

(52) U.S. Cl. CPC A61F 2/5044; A61F 2/80; B29C 70/088; B29L 2031/7532; A61F 2002/5053; A61F 2002/5055; A61F 2002/5056; B29C 70/443

(57) ABSTRACT

A multi-matrix composite socket includes struts formed from a first matrix composite and inner and outer layers relative to the struts formed from at least a second matrix composite. Each matrix composite is comprised of fibers embedded in a resin. The fibers create spaces into which a flowable resin infuses during manufacturing and also reinforce the resin. A base and a plurality of struts are assembled and positioned over a first fabric layer and a second fabric layer is positioned over the struts. A resin is infused into the spaces defined by the first and second fabric layers and around the struts to form an over-molded multi-composite matrix socket.

### CROSS-REFERENCE TO RELATED APPLICATIONS

This application claims benefit to U.S. Provisional Ser. No. 62/736,876, filed Sep. 26, 2018, and U.S. Provisional Ser. No. 62/701,310, filed Jul. 20, 2018, which are hereby incorporated by reference herein in their entireties.

### BACKGROUND

### 1. Field

The present disclosure relates to prosthetic devices and related methods of fabrication.

### 2. State of the Art

The use of prostheses by amputees is well known. Prostheses include a socket to receive a residual limb, a typically modular prosthetic extremity, such as a foot-ankle system, and an interface between the socket and the prosthetic extremity. A variety of sockets and prosthetic extremities are available, which can be combined in any suitable manner to produce a prosthesis that is tailored to meet the individual needs of different amputees.

The socket generally acts as the component of the prosthesis that contains and provides structural support for the residual limb. Specifically, the socket is instrumental as an interface between the residual limb and the prosthetic extremity. For lower limb amputees, the socket is involved in transferring the amputee's weight to the ground by the way of the prosthesis. For upper limb amputees, the socket is the essential component that allows the transfers movement of the residual limb to controlled movement of the prosthetic extremity. If the socket does not fit and operate properly, utility of the prosthesis can be severely compromised. Several factors are considered in the design of a socket, including whether the socket satisfactory transmits the desired load, provides satisfactory stability, provides efficient control for mobility, is easily fitted, and/or is comfortable.

Recent prostheses address these needs using a socket construction including several longitudinal struts, a residual limb interface, a distal base, and an anatomically-shaped fill cup. The struts are connected to a distal base of the socket using a variety of hardware that permits adjustment by the prosthetist for the user. A modular distal extremity also can be adjustably coupled to the distal base. The interface is supported at the interior of the struts and distributes contact and force between the residual limb and the struts. The fill cup is provided at the lower distal end of the interface to maximize support of the residual limb. Such prostheses are described in detail in U.S. Pat. No. 8,978,224 (Hurley et al.), which is incorporated by reference herein in its entirety.

The separate and distinct layers of the interface and the struts add thickness and bulk to the socket of the prosthesis. The thinnest socket is limited to the thinnest struts that are able to provide the necessary radial and longitudinal support to the prosthesis.

### SUMMARY

A prosthetic system includes a socket and a modular distal extremity. For a transtibial prosthesis, the modular distal extremity can include foot and ankle components. The foot and ankle components may also be provided with a tubular pylon element for adjustable attachment to the socket. For a transfemoral prosthesis, the modular distal extremity can include an articulable knee component, as well as foot and ankle components.

The socket of the system includes a weight-bearing structural frame, a residual limb interface, a distal base, and a fill cup. The structural frame defines a receiver with sufficient strength and stiffness to support the residual limb and transfer forces applied from the residual limb to the modular distal extremity. The limb interface is a softer, more form fitting aspect of the socket that is adapted to closely accommodate the anatomical contours and tissue density of the residual limb and/or is adapted to apply/receive force at locations that result in least irritation for the patient and provide the best user results for the prosthesis. The fill cup is an anatomically-shaped soft component provided at the interior of the interface for weight support and weight transfer from the residual limb, through the interface, and to the structural frame.

In accord with one aspect of the system, the structural frame and the interface are integrally molded together as a multi-matrix composite. A matrix is a combination of a polymer resin integrated with fibers. The fibers may be individual strands, fiber bundles, yarns, or cables, or formed as a fabric. The fabrics, by way of example, may be knits, weaves, spacer mesh or open mesh. The fibers create spaces to hold the resin, and the fibers functionally reinforce the resin. A matrix composite is a molded integration of two or more matrices.

The structural frame is preferably comprised of a base and a plurality of longitudinal struts. The base is adapted to support the distal ends of the struts and provides a modular interface with the distal component. The structural frame is made from a first matrix defined by a first resin and first fibers.

The limb interface is a second matrix defined by a second resin and second fibers, such that the second matrix is softer and less rigid than the first matrix. The second matrix is provided at at least one of the interior and exterior sides of the struts. In the second matrix, at least one of, and preferably both of, the second resin and second fibers are different from the first resin and the first fibers. The interface is preferably formed as an inner layer at the radial interior of the struts as well as an outer layer at the radial exterior of the struts. The interface may fully surround the struts or at least partially surround the struts. The first and second matrix together, forming at least the structural frame and the interface, define a multi-matrix composite.

In accord with a method of manufacturing the socket, a positive mold of a residual limb is made. A release agent is provided onto the positive mold. The second fibers (preferably in the form of a second fabric) are placed over the positive mold and the release agent. The structural frame is then placed onto the mold over the second fabric. Third fibers (preferably in the form of a third fabric) is then placed over the structural frame. A vacuum bag, having at the inside thereof a release agent, is positioned over the third fibers.

A curable liquid resin is provided into the vacuum bag and a vacuum source is applies negative pressure to the interior of the system to draw the resin through the third fabric, about the assembled struts and base, and through the second fabric. The resin is allowed to cure. Then, the socket is removed from the mold. Other manufacturing methods, including layered thermoplastic sheets, can also be used.

The constructed socket includes the first matrix of the structural frame at least partially sandwiched between and within the second matrix of the interface. The fill cup is provided within a cavity defined at the interior of the interface and functions as a softer, lower durometer, boundary layer between the residual limb and the remainder of the socket.

Additional matrix layers can be added to the socket construction. For example, additional struts, strips, bands, panels, etc. that are premanufactured fiber resin matrices can be inserted between structural frame and the interface or within a portion of the interface to define areas of intermediate or greater rigidity. Further, fabric strips, fabric bands, fabric panels, etc. can be inserted between the structural frame and the layers of the interface and the vacuum bag to define areas of greater rigidity or which have different properties once the resin permeates therein.

Non-matrix layers and/or components can be added to the construction. For example, stiffening plates, closures, guideways and channels, housing, retention mechanisms including portions of vacuum systems, pin lock systems, magnetic latching systems, and buckle systems, adjustment systems, sensing systems, and communications systems can be molded integral with the socket by positioning the components on the mold and in relation to the appropriate layer during the molding process.

After removal from the positive mold, the multi-composite matrix socket can be finish-shaped by cutting, grounding, sanding, buffing or otherwise shaping to accommodate necessary components and receiving the residual limb. Further, because the composite matrix is thermoplastic, the socket alternatively or additionally can be heated for shaping (and re-shaping) to better accommodate receiving, supporting and interfacing with the residual limb.

### BRIEF DESCRIPTION OF THE DRAWINGS

FIG. 1 shows a patient with an amputation and wearing a prosthesis with a prosthetic socket according to the system and method.

FIG. 2 is a plan view of a base component of a structural frame for the prosthetic socket.

FIG. 3 is a side elevation view of the base component of the structural frame.

FIG. 4 shows the side by side disassembled components of a multicomponent base component.

FIGS. 5 and 6 are flow charts illustrating an exemplar manufacture of the prosthetic socket.

FIGS. 7 and 8 show steps in the manufacture of the structural frame of the prosthetic socket.

FIG. 9 is a broken, partial transparent inverted view (in the position formed on the mold) of the multi-composite matrix prosthetic socket described herein.

FIG. 10 through 21 show steps in the manufacture of a prosthetic socket according to a method described herein.

### DETAILED DESCRIPTION

For the sake of convenience, much of the following disclosure is directed to prosthetic systems that are configured for use with a residual portion of an amputated leg, such as a leg that has undergone a transfemoral (i.e., above-knee) or transtibial (i.e., below-knee) amputation. It should be appreciated that the disclosure is also applicable to other prostheses, such as those configured for use with the residual limb of an amputated arm (e.g., after an above-elbow or below-elbow amputation).

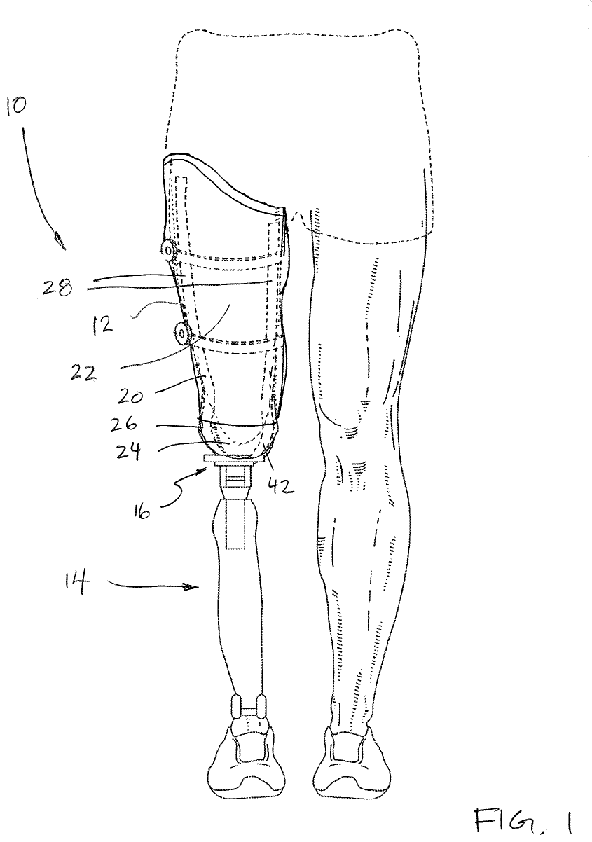

Referring to FIG. 1, a prosthetic system 10 includes a socket 12 and a modular distal extremity 14. For a transtibial prosthesis, the modular distal extremity 14 can include foot and ankle components. The foot and ankle components may also be provided with a tubular pylon element and couplers (not shown) for adjustable attachment to the socket 12. For a transfemoral prosthesis, the modular distal extremity can include an articulable knee component 16, as well as foot and ankle components.

The socket 12 of the system includes a weight-bearing structural frame 20, a residual limb interface 22, and a fill cup 24. The structural frame 20 defines a longitudinally extending receiver that has sufficient strength and stiffness to support the residual limb and transfer force applied from the residual limb to the modular distal extremity. The limb interface 22 is of a softer durometer, and more form fitting than the frame such that it is adapted to more closely accommodate the anatomical contours and tissue density of the residual limb and/or is adapted to apply/receive force at locations that result in least irritation for the patient and provide the best user results for the prosthesis. The fill cup 24 is an anatomically-shaped, lower durometer component provided at the interior of the interface 22 for weight support and weight transfer from the residual limb, through the interface 22, to the structural frame 20.

In accord with one aspect of the system 10, the structural frame 20 and the interface 22 are integrally molded together as a multi-matrix composite. For purposes of the following description and claims, and as described in more detail below, a matrix is a combination of a polymer resin integrated with fibers. By way of example only, the resin may be a polymethylmethacrylate (PMMA) or a polyurethane. The fibers may be individual strands, fiber bundles, yarns, or cables, or formed as a fabric. The fiber, by way of example only, may be natural fibers such as cotton fibers, or manufactured fibers such as carbon fibers, nylon fibers, rayon fibers, NY-GLASS, POLY-GLASS, and highly elastic nylon fibers such as sold under the tradename EXTENDA, and/or heat and flame-resistant fibers such as synthetic aromatic polyamide polymer, e.g., sold under the tradename NOMEX (Dupont). The fabrics, by way of example, may be knits, weaves, spacer mesh or open mesh. The fibers create spaces to hold the resin, and the fibers functionally reinforce the resin. The fibers can also create decorative or mechanically useful patterns on the surface of the cured resin. The fibers can also create decorative colors patterns within the interior of the cured resin, particularly where the cured resin is transparent or translucent. A matrix is defined as being different from another matrix if it comprises different a resin, a different Shore hardness, a different modulus of elasticity, a different color of a same resin, a different cured thicknesses of the same or different resin, different fiber materials, different dimensions of the same or different fibers (i.e., different fiber diameters or substantially different cut lengths), different fabric constructions of the same or different fibers (knits, weaves, or meshes of fibers), and/or any combination of the previous. A composite matrix is a molded integration of two or more matrices.

The structural frame 20 is preferably comprised of a base 26 and a plurality of longitudinal struts 28. In one embodiment, the struts 28 are provided as relatively flat, separate and distinct longitudinal components. The struts 28 are made from a first matrix of a resin and fibers. The first matrix has thermoplastic properties such that, on heating to a defined deformation temperature, it becomes plastic and can be reshaped; subsequently, on cooling it re-hardens. The first matrix is able to have these processes repeated. In accord with a preferred construction, the first matrix includes a polymethylmethacrylate resin and carbon fibers retained with the resin.

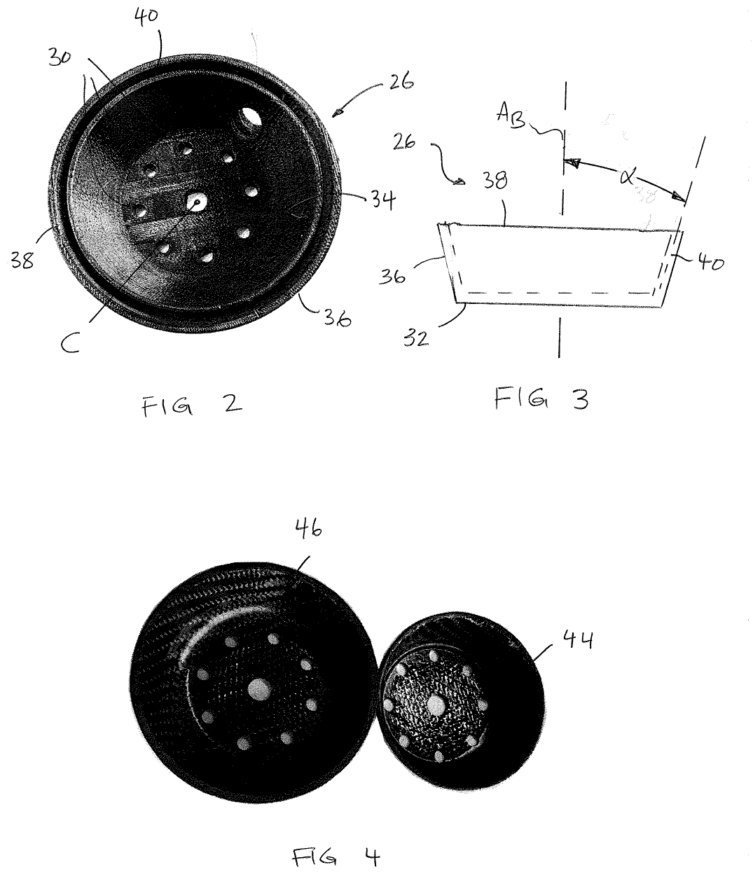

Turning also to FIGS. 2 and 3, the base 26 is adapted to support the struts 28 and provides a modular interface, such as a hole pattern 30, at its distal end 32 for connecting to the distal extremity 14. The base 26 is preferably made from the first matrix, but may be made from another matrix. The base 26 has a radial center C defining a central axis A_{B}. In one embodiment, the base 26 is a hollow frustoconical member with inner and outer surfaces 34, 36 spaced apart at its larger-diameter, proximal end 38 to define a slightly radially outwardly angled α) (0°-30°) channel 40 sized to snugly receive the distal ends 42 of the struts 28 in a circumferentially spaced apart relationship. The channel 40 may be a single circumferential channel or may be a plurality of channels adapted to receive one or only a select number of strut distal ends about the periphery of the base 26 between the inner and outer surfaces 34, 36. The base 26 may be a unitary component. Alternatively, as shown in FIG. 4, the base 26 may be comprised of inner and outer generally bowl-shaped components 44, 46 that can be nested one within the other, with the channel(s) 40 defined as the space between sidewalls of the nested components. The inner and outer components 44, 46 can be of the same wall thickness or the wall thickness on the outer component 46 can be thicker. The forces on the struts 28 that will be retained at the base 26 will be radially outward; thus, higher forces will be applied to the outer component 46. An inventory of different base units or assemblies can be provided to permit construction of the base and struts in a desired size and configuration. Unitary bases or components for manufacturing bases are provided which permit construction of bases at different diameters, proximal-distal wall heights, wall thicknesses, mounting locations, and channel angle. By way of example, bases 26 can be provided that have one or more channels 40 oriented with α=0°, 5°, 10°, 15°, 20°, 25° or 30° relative to the base axis A_{B}. As yet another alternative, the base 26 and struts 28 may have different respective structure to permit spaced apart engagement of the struts about the periphery of the base. The distal ends 42 of the struts 28 are coupled to the periphery of the base 26, i.e., in the channel(s) open at the proximal end of the base 26. The struts 28 are coupled to the base by adhesion, by bonding, or by mechanical coupling. While the struts 28 are preferably provided as separate and distinct elements, they can be partially or fully integrated, or even initially molded, cut, bent, or otherwise manufactured integral with the base as a preformed structural frame 20.

According to another embodiment, the base 26 and struts 28 are provided separately and applied separately during the socket molding process. In such embodiment, the base 26 and struts 28 can be assembled relative to the socket at different steps during a socket molding procedure, described below, and the struts can be separately heat formed, cut, and bent relative to a mold without interference from other integrated struts or the base.

The interface 22 is a second matrix defined by a second resin and second fibers provided at at least one of the interior and exterior sides of the struts. In the second matrix, at least one of, and preferably both of, the second resin and second fibers are different from the first resin and first fibers. More preferably, the interface is formed as an inner layer at the radial interior of the struts as well as an outer layer at the radial exterior of the struts. The interface 22 may fully surround the struts 28 or at least partially surround the struts 28. By “partially surround” it is meant that the interface may partially, but not fully, cover portions of a plurality of struts, as well as the interface fully surrounds at least one of the struts and is absent from at least one of the struts. By “at least partially surround” it is meant that the interface may be located in relation to the struts as defined by either “partially surround” or further surround the struts up to what is commonly considered to fully surround or to fully encompass the struts. The second matrix is softer and less rigid than the first matrix. The first and second matrix together, forming at least the struts and the interface, define a multi-matrix composite with properties of both the first and second matrices.

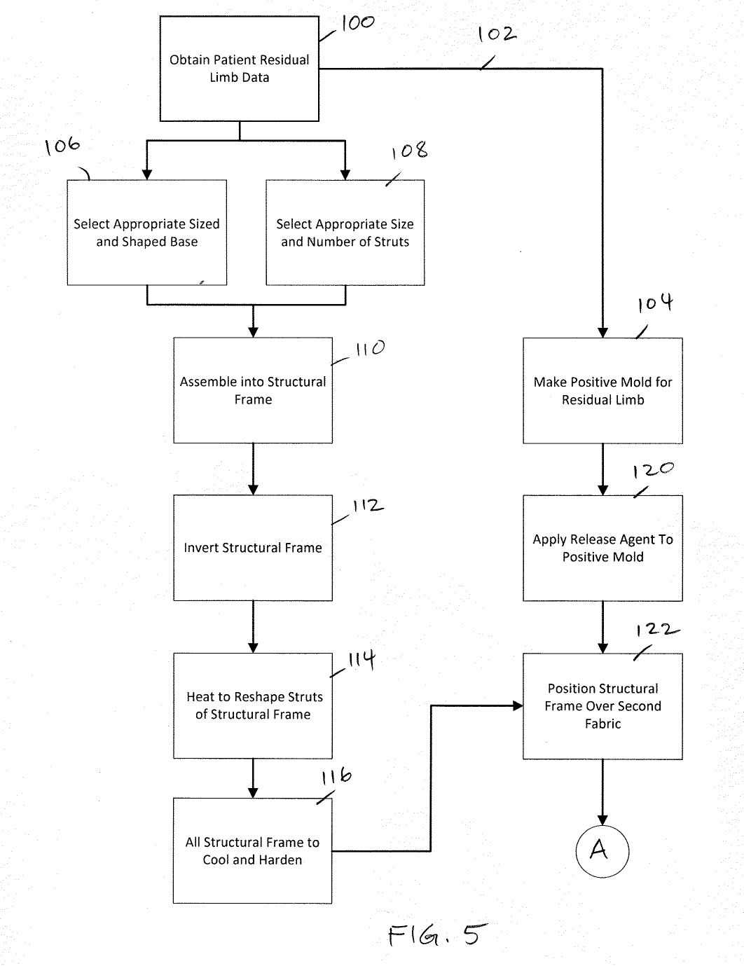

Turning now to FIGS. 5 and 6, in accord with one method of manufacture of a prosthetic device, data regarding the patient residual limb is obtained at 100. Such data may be obtained from direct measurement, photogrammetry, sensing, scanning, or indirectly from a digital or physical model of the residual limb.

Once the size and shape of the residual limb are measured or otherwise determined and the needs of the patient have been analyzed, a structural frame is made or otherwise provided for use in manufacture of the socket 102, and an appropriate physical model of the residual limb is made at 104 and as discussed below.

The structural frame is manufactured for the patient at 102. In such manufacture, an appropriate base and struts are required for the patient. The base and struts are selected from an inventory of bases and struts. The base may be selected at 106 on the type of prosthesis, the size and weight of the patient, the defined channel angle, and the struts to be attached thereto. The struts may be selected at 108, e.g., in length and/or thickness, based on the type of prosthesis, the size and weight of the patient, and/or the location (anterior, posterior, medial, lateral, or some intermediate position) at which the strut is to be attached to the base. The distal ends of the struts are inserted into the channel at the proximal end of the base, and secured thereto at 110. They are preferably secured with an adhesive that, once cured, has a higher melting point that the melting point of the first matrix.

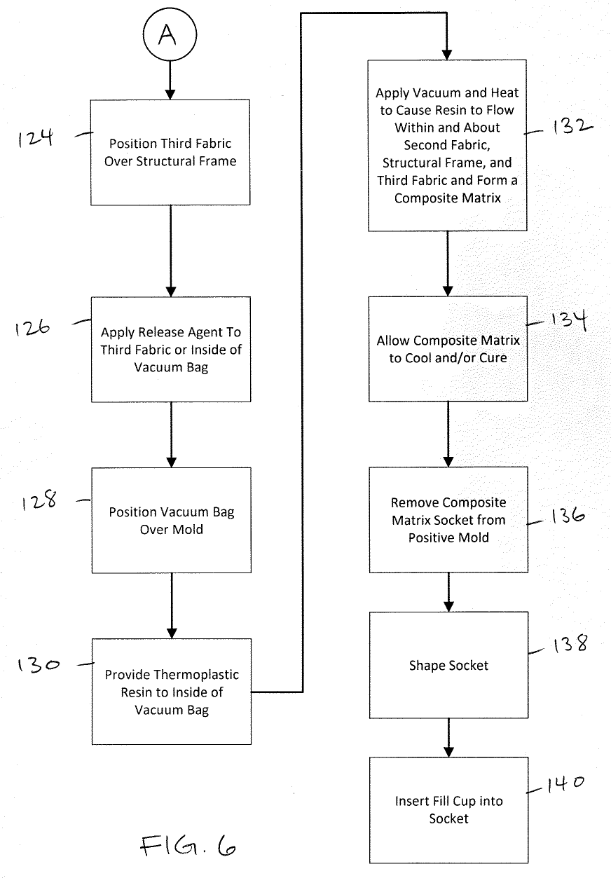

In one step of the method, the base with straight struts is inverted at 112 (such that the struts 28 extend downward from the base 26, as shown in FIG. 7). Because the channel 40 extends outward at an angle, the straight struts 28 are angled outward by the angle of the channel. The base 26 and struts 28 are then heated at 114 above a sufficient temperature to soften the struts 28 and, as a result of the weight of the struts and the flexibility of the heated struts, the struts extend vertically downward parallel to the axis A_{B} of the base 26 and define an angled-bend 48 where exiting the channel 40, as shown in FIG. 8. Heat is then reduced, and the struts are permitted to re-harden and fix their position in relation to the base at 116.

In another step of the method, the positive mold of the residual limb is made at 104. The positive mold may be made from any suitable process. By way of example, first a cast of the residual limb may be made by wrapping the limb in a fabric provided with a preferably non-caustic first molding material. The fabric and first molding material may include a fabric provided with plaster of Paris, or a fabric provided with a low temperature curing polymer, or other suitable fabrics and first molding materials. After curing, the cured first material which covered the appropriate portion of the residual limb for the socket is carefully removed from the residual limb to define at its interior surface a negative mold in the shape of the residual limb. The negative mold is then filled with a curable second material and the second material is allowed to cure. Once the second material is cured, the negative mold is removed from over the cured second material to expose the positive mold.

In accord with another example for manufacture of the positive mold, data representing the three-dimensional contours of the residual limb is used in association with a CNC system to cut the positive mold from a block of suitable material, including plaster, plastic, wood, etc. In accord with yet another exemplar method, data representing the three-dimensional contours of the residual limb is used to print in three dimensions the positive mold from one or more suitable stock material(s). Other methods, including combinations of the methods, can be used.

Next, a release agent is provided onto the positive mold at 120. The release agent is adapted to assist in removal of the cured socket (described below) from over the mold.

A second fabric comprised of second fibers is placed over the positive mold and the release agent at 122. The second fabric is an eventual component of the second matrix which will overmold with the base and struts. The second fabric may be in the form of an open-ended sock, bands, strips, panels, or any other generally flat fiber-form.

The assembled unit of the base and struts 20 is then positioned at 122 over the second fabric and onto the mold in accord with a desired alignment relative to the mold.

A third fabric comprised of third fibers is then placed at 124 over the structural frame. The second and third fabrics may be the same or different, depending on whether the interface is intended to have different properties at its interior and exterior.

A vacuum bag, having at the inside thereof a release agent 126, is positioned over the third fabric at 128. A resin is released to, poured into, injected into or otherwise provided to the interior of the vacuum bag at 130. A vacuum source coupled to the vacuum bag is activated to apply at 132 a negative pressure to the interior of the system to draw the resin through the third fabric, about the assembled struts and base, and through the second fabric. In addition, external pressure is preferably also applied about the outside of the vacuum bag and onto the system by wrapping the system tightly in a compression member such as a fabric wrapping, a belt, a band, elastics, etc. The resin is allowed to cool or otherwise cure at 134 to form a composite matrix that includes the structural frame. Then, the vacuum bag is removed from over the cured resin. The socket is removed from the mold at 136.

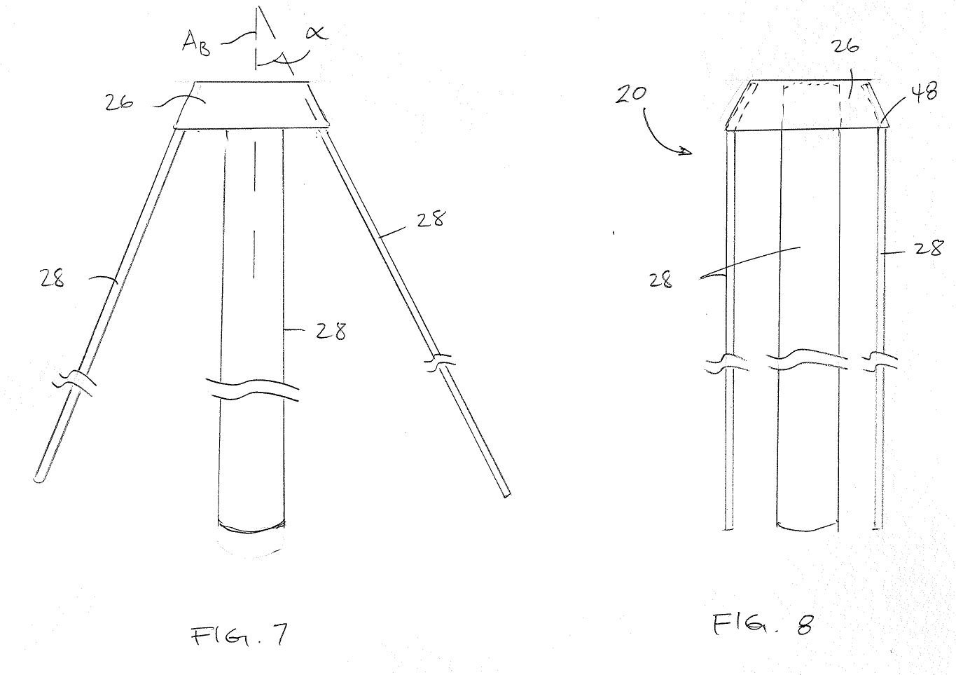

From the above, turning to FIG. 9, the resulting socket 12 includes the following portions. The struts 28 are made from fiber resin matrix and define a first matrix. The second fibers of the second fabric at 50 and the second resin define the second matrix. The third fibers of the third fabric at 52 and the second resin define a third matrix, which may be the same as the second matrix, or may be different from the second matrix if the first and second fibers are different from each other. Thus, the inner layer of the socket comprises the second matrix, the middle layer of the socket comprises the first matrix, and the outer layer of the socket comprises the third matrix. The fill cup is provided within the cavity defined at the interior of the inner layer to function as a softer boundary interface between the residual limb and the remainder of the socket.

In addition, second and third resins can be different from each other by modifying the process the change the resin part way through the socket molding. In such manner, the second resin is applied earlier in the process and is drawn to a deeper layer, i.e., the inner layer, and third resin is applied later in the process and is at the outside of the socket. For example, it may be advisable to use a softer more accommodating resin at the inner layer, and a stiffer resin at the outer layer.

Further, additional matrix layers can be added to the socket construction. For example, additional struts, strips, bands, panels, etc. that are premanufactured composite matrices can be inserted between the second fabric and the struts, or between the third fabric and the vacuum bag to define areas of intermediate or greater rigidity. By way of further example, fabric strips, fabric bands, fabric panels, etc. can be inserted between the struts and the first fabric, or the second fabric and the vacuum bag to define areas of greater rigidity or which have different properties once the resin permeates therein.

Moreover, non-matrix layers and/or components can be added to the construction. For example, metal stiffening plates can be molded in the structure. By way of another example, closures 60, guideways and channels 62, lacings, housings for components, adjustments, sensors, pressure applicators, adjustable or fixed fluid bladders, magnets, mechanical receivers, release mechanisms, drainage ways, displays, electronic signal transmitters and/or receivers, etc. can be molded integral with the socket by positioning the components on the mold and in relation to the appropriate layer during the molding process.

After removal of the socket from the positive mold, the multi-composite matrix socket can be finish shaped at 138 by cutting, grinding, sanding, buffing or otherwise shaping to accommodate receiving the residual limb. Further, because the composite matrix is thermoplastic, the socket alternatively or additionally can be heated for shaping (and re-shaping) to better accommodate receiving, supporting and interfacing with the residual limb.

Moreover, even after molding and before or after finish shaping the socket may be provided with closure components, including tension elements such as straps, bands, and cables, and housing and/or routings for such tension elements, all to modify the shape the socket to facilitate retention of the socket on the residual limb and adjustment of the socket to the residual limb. Also, the socket may be adapted or modified to allow the socket to accommodate other types of socket retention mechanisms including vacuum systems, pin lock systems, magnetic latching systems, buckle systems, etc., adjustment systems, sensing systems, and communications systems.

After manufacture of the composite matrix portion of the socket, the fill cup 24 is inserted at 140 into the interior cavity of the socket.

In accord with an alternative manufacture process, in which an initially flowable resin for the second and third matrices is not required, thermoplastic sheets are provided over the positive mold at the interior and exterior of the structural frame. Heat and vacuum are then applied to cause the sheets to flow, melt, mold or otherwise form about the structural frame. The thermoplastic sheets may be pre-provided as matrices with a fabric or fiber layer therein. Alternatively, the second fabric layer is provided between an inner thermoplastic sheet and the structural frame and a third fabric layer is provided between the structural frame and the outer thermoplastic sheet. Then, upon application of heat and vacuum, the thermoplastic sheet materials also flow within the second and third fabrics and form respective matrices. Optionally, the adjacent surfaces of one or both of the inner and outer thermoplastic sheets may be surface roughened to facilitate adhesion between the sheet layers. Further optionally, an adhesive may be applied to one or both of the outer surface of the inner thermoplastic sheet, the inner surface of the outer thermoplastic sheet, the inner surface of the structural frame, or the outer surface of the structural frame.

In accord with another method of manufacture of a prosthetic device, data regarding the patient residual limb is obtained and a positive mold of the residual limb is made, both in accord with any method described above.

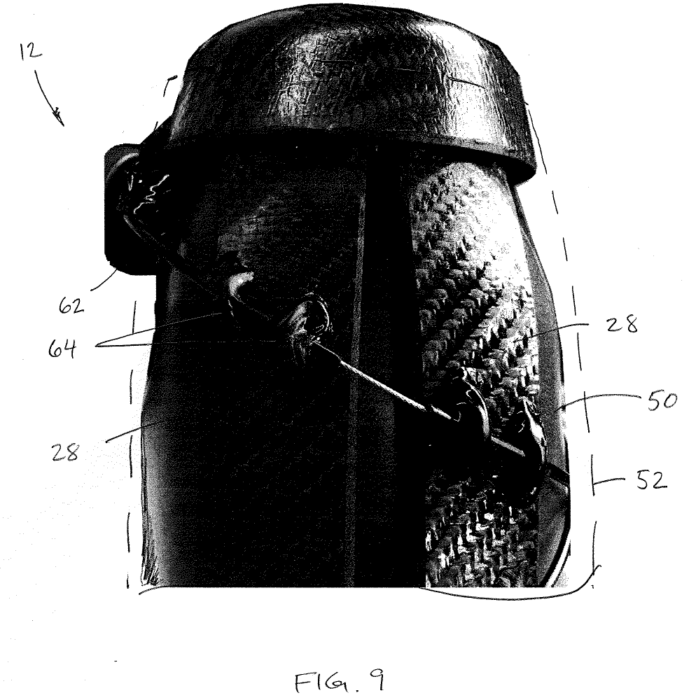

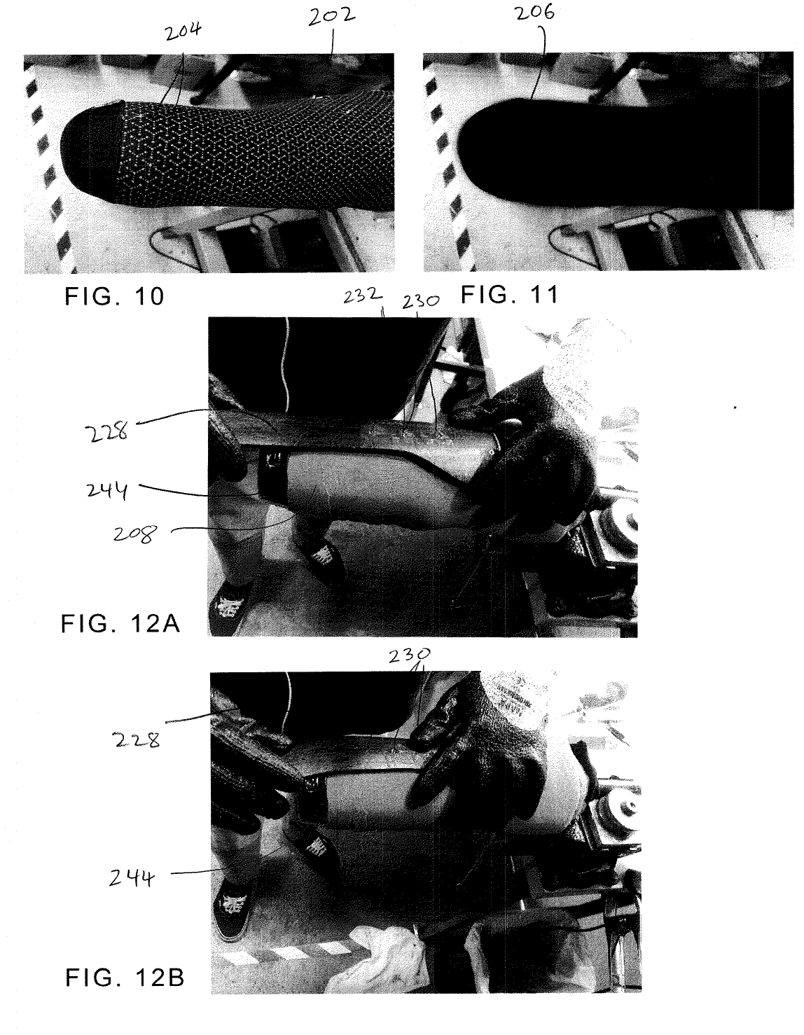

Then, as shown in FIG. 10, a molding fabric 202 having a raised design intended to be impressed onto the inner surface of the socket is provided as a sock-type layer over the mold. (This inner fabric also can be used over the mold in the previously described method, as well.) One such preferred fabric has a plurality of raised, small, closely and evenly spaced, Y-shaped designs 204 positioned over the surface over the fabric to, in turn, provide a negative impression of such Y-shaped designs onto the inner surface of the socket in accord with a process described below. The molding fabric 202 forms no part of the actual prosthetic socket. A PVA polymer bag (not shown) is pulled snugly over the molding fabric and mold and forms a separation barrier between the molding fabric and the socket to be manufactured.

Turning to FIG. 11, an inner sock layer 206 defining a second fabric is snugly pulled over the PVA bag. In an embodiment, the second fabric is a cotton or a cotton blend. Optionally, one or more structural components may be placed over the second fabric. A third fabric, preferably in the form of a middle sock layer 208, is provided over the second fabric (shown in FIGS. 12A-12B). In accord with a preferred aspect of the method, the third fabric 208 is a temperature tolerant, heat resistant layer, that can be subject to relatively high temperatures; i.e., up to and in excess 400°.

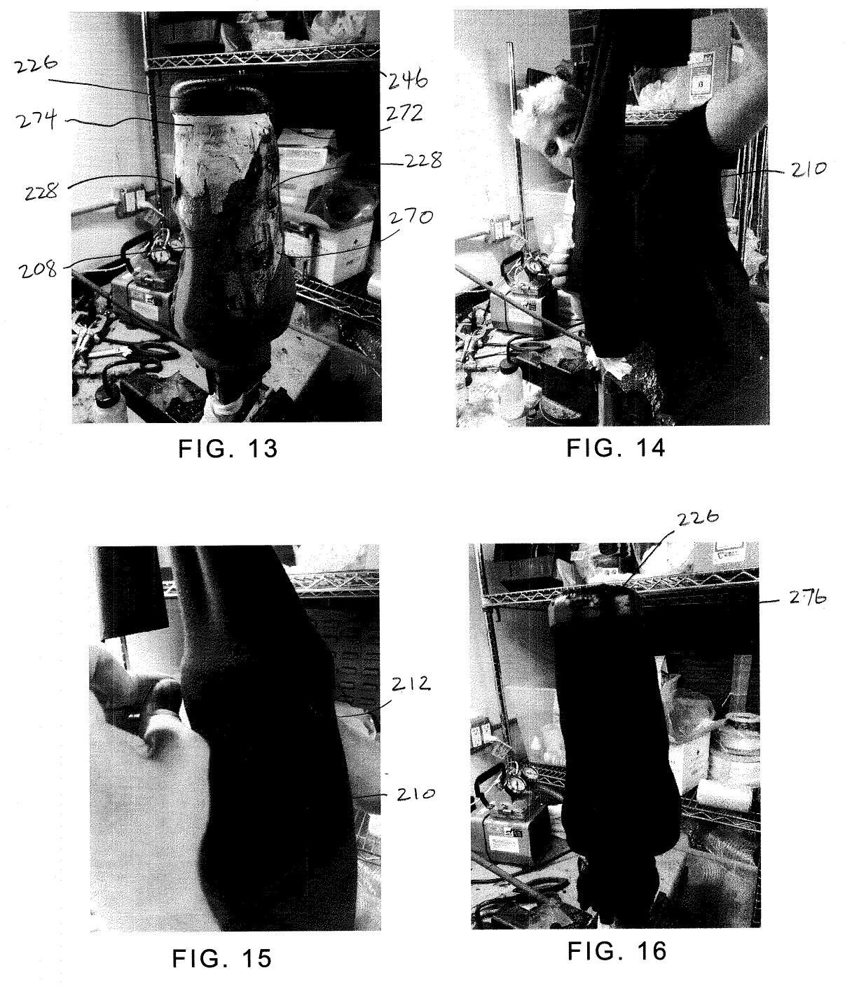

Referring to FIGS. 12A and 12B, the structural frame is then formed on top of the third fabric 208. In distinction from the earlier method, the frame is positioned onto the mold as separate components. The frame comprises the struts 228, an inner cup 244, and an outer cup 246 (FIG. 13). The components of the frame are made of the same materials as the structural frame described above; that is, the frame is constructed from a first fabric saturated with a first resin to form a first matrix. The inner cup portion 244 of the base, optionally selected from a store of inner cup portions, is provided over the distal end of the fabric-covered mold, covering the distal end of the third fabric 208. The struts 228 are then selected from a store of struts of different sizes and/or shapes. The struts 228 preferably include an arrangement of holes 230, each provided with a threaded collar 232 retained at an inner surface of the strut and/or within the hole (at least once retained on the mold). The struts 228 are heated in a heat press, with a heat gun, or with another heating device, and then positioned onto the mold over the third fabric 208. In the heated condition, the struts 228 can be molded by hand and/or bending or forming tool to conform to the mold, as shown in FIG. 12B. Such conformation does not require that the struts exactly follow the contours of the mold, although they can; rather, conformation requires that the struts be bent, as necessary, in accord with the judgment of the prosthetist or technician to accommodate the patient once the socket is complete. A jig may be used to assist in positioning the angular orientation of the struts and/or holding the struts while they are molded relative to the mold. The struts 228 may be molded in a set sequence, in any order, or portion-by-portion of each of several until the desired shape of the skeletal frame is complete. Then, the outer cup portion 246 of the base of appropriate size is selected from a store of components, and provided over the distal end of the mold, capturing the distal ends of the struts 228 between the inner and outer distal cups 244, 246 (FIG. 13). A primer 270 is preferably painted, sprayed or otherwise applied onto the surface of the struts 228. The primer 270 assists in adhesion of the thermoplastic resin to the struts.

It has been identified that a physical step 272 forms between the proximal end of the outer distal cup 246 and the surface of the third fabric layer 208. This physical step presents a substantially large space for thermoplastic resin to enter and fill in the subsequent stage of the manufacturing procedure. The resin is relatively dense and, if filled into all such space would result in a socket that is heavier than desirable. Therefore, in accord with one aspect of the method, a void filler 274 is filled into spaces present adjacent the base 226 as well as over selected distal portions of the third fabric 208. The void filler 274 is an expandable, relatively low-density, hardenable foam that is adapted to occupy space otherwise fillable by a thermoplastic second resin to reduce weight of the socket.

Turning to FIG. 14, an outer sock layer 210 of a fourth fabric is pulled down over the above-described construct, and then reflected back up over the mold to provide a dual layer of the fourth fabric on the mold assembly. In an embodiment, the fourth fabric is a cotton fabric. The outer sock layer 210 is tied off with a thread, string, wire, cable or other suitable elongate structure 212 to tie define a hard edge at its border with the base 226 (FIG. 15). The base 226 is taped off with tape 276 to protect its outer surface during the molding process (FIG. 16).

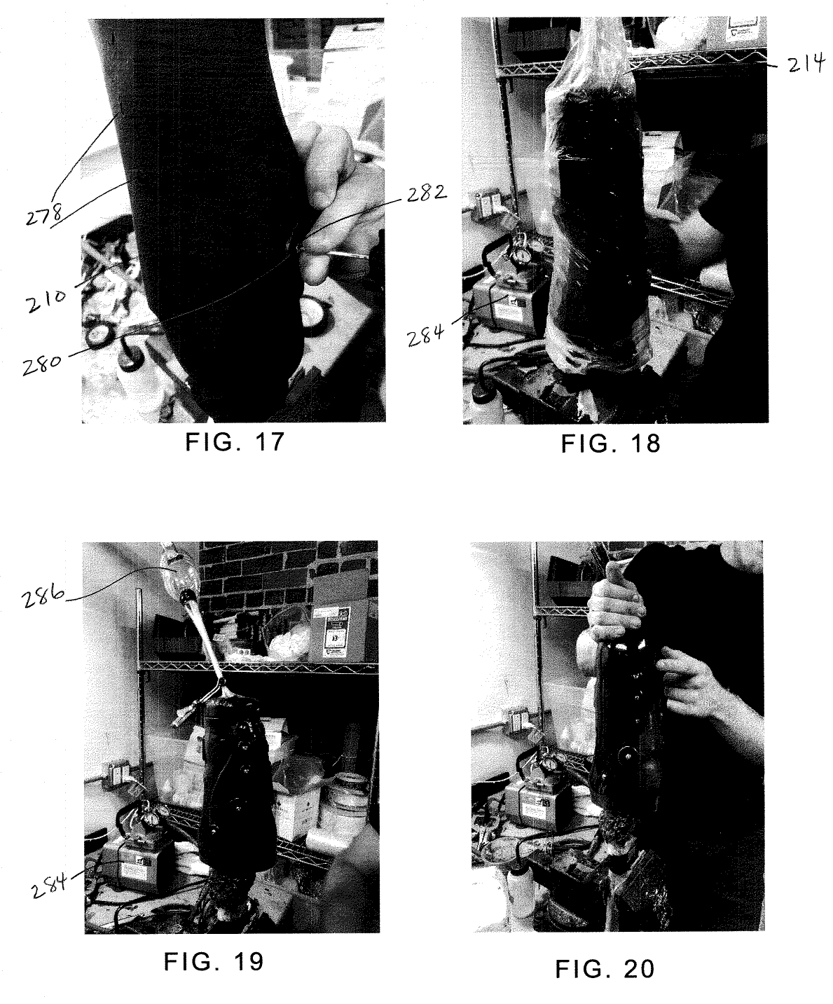

As shown in FIG. 17, holes 278 are cut into at least the outer sock layer 210 over the holes 230 in the struts. Stiff, round, planar washers 280 are attached to the outside of the outer sock layer 210 over the struts 228 with retaining screws 282 that threadedly engage the collars in the holes 230. The washers 280 may be of various sizes in order to accommodate mounting particular structure, as discussed below.

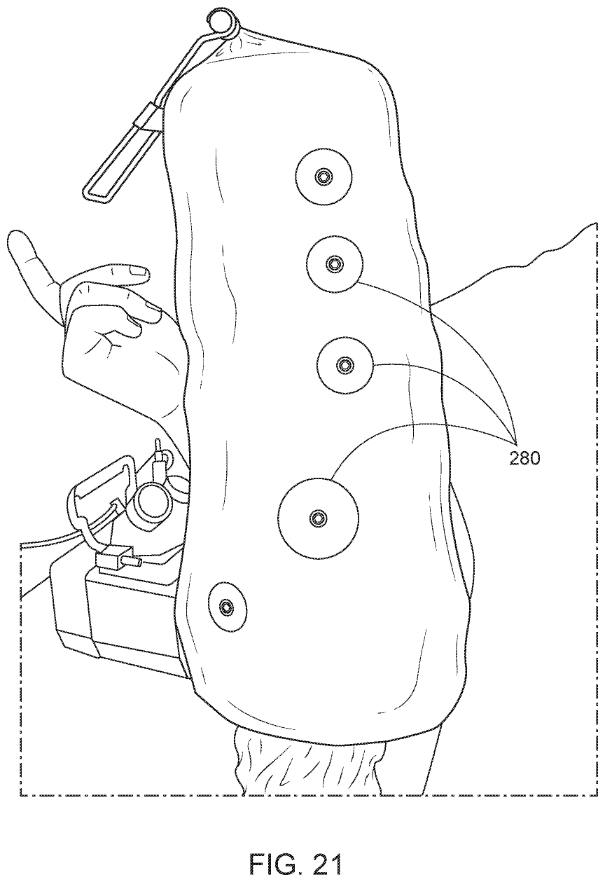

Then, referring to FIGS. 18 and 19, an outer PVA bag 214 is pulled over the assembly and coupled to a vacuum source 284 at its lower end, proximate the proximal end of the socket, and coupled to a flowable thermoplastic second resin feed source 286 proximate the distal end of the socket. With vacuum applied to draw the bag 214 against the mold assembly, the thermoplastic second resin is fed from the feed source 286 between the inner and outer PVA bags and drawn down about the mold assembly, surrounding the struts, and saturating the three sock-like layers of fabric positioned on the mold. Manual force may be applied about the outer PVA bag 214 and the flowing thermoplastic resin within bags to squeeze and spread the second resin evenly and/or completely over the assembly (FIGS. 20 and 21). The second resin is allowed to sufficiently cure. Then, the outer PVA bag is removed.

The second resin is cleanly cut at the elongate structure 212 tying off the base 226. The tape 276 is removed from over the base 226, providing a clean appearance to the base.

The areas of the socket provided with void filler 274 have a reduced density in relation to the areas of the socket with thermoplastic resin; thus, the void filler operates to significantly reduce the weight to the socket.

A shown in FIG. 21, the washers 280 provide consistent, flat, molded-in mounting locations on the exterior of the otherwise contoured socket for mounting closure, adjustment, cable guidance structures on the socket. The closure or guidance structure can include a central or offset hole, and the retaining screw in the washer can be removed, inserted through the hole, and again secured relative to the threaded collar at the mounting location. The round shape of the washers provides a consistent shape regardless of the orientation of the closure or cable guidance structure. Other structures may also be mounted at the mounting locations, including channel elements, lacings, sensors, pressure applicators, fluid bladders, magnets, electronic signal transmitters, electronic signal receivers, release mechanisms, drainage ways, and displays.

The first and second resins may be the same or different from each other. The fabric may be the same or different from each other. However, in accord with the composite resin matrix aspect of the described system and method, at least one of the fabrics and/or resins should be different from another.

There have been described and illustrated herein embodiments of a system and methods of manufacturing the system. While particular embodiments have been described, it is not intended that the invention be limited thereto, as it is intended that the invention be as broad in scope as the art will allow and that the specification be read likewise. It will therefore be appreciated by those skilled in the art that yet other modifications could be made to the provided invention without deviating from its scope as claimed.

What is claimed is:

1. A method of manufacturing a prosthetic socket for a residual limb of a patient, comprising:
a) providing a structural frame having a first modulus, the structural frame comprised of a first matrix of a first thermoplastic resin and first fibers;
b) providing a mold of the residual limb;
c) positioning an inner layer of second fibers onto the mold;
d) positioning the structural frame onto the mold over the layer of second fibers;
e) positioning an outer layer of third fibers onto the mold over the structural frame; and
f) causing a thermoplastic second resin to flow within the second and third fibers and around the structural frame to form a composite matrix including the inner layer, the first matrix of the structural frame, the outer layer and the second resin.

2. The method of claim 1, wherein providing the structural frame includes assembling the structural frame from a base and a plurality of struts.

3. The method of claim 2, wherein both of the base and the struts are made from the first matrix.

4. The method of claim 1, further comprising placing a vacuum bag over the outer layer of third fibers, and wherein the causing the second resin to flow includes applying negative pressure at an interior of the vacuum bag.

5. The method of claim 1, wherein before causing the second resin to flow, further comprising placing a sheet of the second resin between the structural frame and the mold.

6. The method of claim 1, wherein before causing the second resin to flow, further comprising placing a sheet of the second resin over the outer layer of third fibers.

7. The method of claim 1, wherein the second fibers are different than the first fibers.

8. The method of claim 1, wherein the third fibers are different than the first fibers.

9. The method of claim 1, wherein the first fibers, the second fibers and the third fibers are different from each other.

10. The method of claim 1, wherein the second fibers and the third fibers are provided as separate fabrics.

11. The method of claim 1, further providing a third resin, wherein the second and third resin are integrated together by heating.

12. The method of claim 1, further comprising providing at least one of:
i) a closure,
ii) a guideway,
iii) a channel,
iii) a lacing,
iv) a sensor,
v) a pressure applicator,
vi) a fluid bladder,
vii) a magnet,
viii) an electronic signal transmitter;
ix) an electronic signal receiver;
x) a release mechanism,
xi) a drainage way, and
xii) a display,
within the formed composite matrix.

13. The method of claim 1, further comprising molding a plurality of planar mounting platforms, each with a hole therein, into the formed composite matrix.

14. The method of claim 1, further comprising positioning an intermediate layer of heat resistant fibers between the inner layer of second fibers and outer layer of third fibers, wherein at least a portion of the structural frame is located on the intermediate layer.

15. The method of claim 1, further comprising adding a primer to a surface of at least a portion of the structural frame.

16. A prosthetic socket for accommodating and supporting a residual limb of a user, comprising:
a) a structural frame having a proximal end, a distal end, an interior side, and an exterior side, and defining a space within the exterior side sized to receive the residual limb, the structural frame formed from a first matrix of a first resin and first fibers;
b) an inner interface layer at at least a portion of the interior side of the structural frame, the inner interface layer formed from a second matrix of a second resin and second fibers; and
c) an outer interface layer at at least a portion of the exterior side of the structural frame, the outer interface layer formed from a third matrix of a third resin and third fibers, wherein
at least one of the second and third resins is different from the first resin, and
at least one of the second and third fibers is different from the first fibers, such that the inner interface layer, the structural frame, and the outer interface layer together define a composite matrix.

17. The prosthetic socket of claim 16, wherein the second fibers of the inner interface layer comprise a multilayer of fibers nearer the structural frame and fibers further from the structural frame, the nearer and further fibers different from each other and the first fibers.

18. The prosthetic socket of claim 16, wherein the second fibers comprise heat resistant fibers.

19. The prosthetic socket of claim 16, further comprising a plurality of a mounting platforms, wherein the outer interface layer is contoured and the mounting platforms are each flat and include a hole adapted for mounting a closure element, fit adjustment element, or cable guide element.

20. A prosthetic socket for accommodating and supporting a residual limb of a user, comprising:
a) a structural frame having a proximal end, a distal end, an interior side, and an exterior side, and defining a space within the exterior side sized to receive the residual limb, the structural frame formed from a first matrix of a first resin and first fibers;
b) a first interface layer at a first side of the structural frame, the first interface layer formed from a second matrix of a second resin and second fibers; and
c) a second interface layer at a second side of the structural frame, the second interface layer formed from a third matrix of a third resin and third fibers,
wherein the second resin is different from the first resin such that the first interface layer, the second interface layer, and the structural frame together define a composite matrix.

21. A prosthetic socket for accommodating and supporting a residual limb of a user, comprising:
a) a structural frame having a proximal end, a distal end, an interior side, and an exterior side, and defining a space within the exterior side sized to receive the residual limb, the structural frame formed from a first matrix of a first resin and first fibers;
b) a first interface layer at a first side of the structural frame, the first interface layer formed from a second matrix of a second resin and second fibers; and
c) a second interface layer at a second side of the structural frame, the second interface layer formed from a third matrix of a third resin and third fibers,
wherein the second fibers are different from the first fibers such that the first interface layer, the second interface layer, and the structural frame together define a composite matrix.
